(19) United States
(12) Patent Application Publication
(10) Pub. No.: US 2021/0196975 A1
(43) Pub. Date: Jul. 1, 2021

(54) LIGHT THERAPY APPARATUSES AND METHODS

(71) Applicant: LiteCure, LLC

(72) Inventors: Luis De Taboada, Carlsbad, CA (US); James Harrison, Oro Valley, AZ (US); Brian F. Swienton, Dover, NH (US)

(73) Assignee: LiteCure, LLC, Newark, DE (US)

(21) Appl. No.: 16/338,778

(22) PCT Filed: Jul. 31, 2017
(86) PCT No.: PCT/US2017/044586

§ 371 Date: Apr. 2, 2019

Related U.S. Application Data
(60) Provisional application No. 62/369,158, filed on Jul. 31, 2016.

(51) Int. Cl. A61N 5/06; A61H 9/00

(52) U.S. Cl. CPC A61N 5/0616; A61H 9/0078; A61N 2005/067; A61N 2005/0647; A61N 2005/0651; A61N 2005/0626

(57) ABSTRACT

Light therapy systems described herein are configured to by worn by or otherwise affixed to the user so that the user is ambulatory while receiving light therapy. In some implementations, one or more physiological sensors are used such that delivery of the light therapy is effected by physiological parameters of the user detected by the physiological sensors. Moreover, in some implementations the apparatuses described herein also deliver compression therapy in addition to the light therapy.

### CROSS REFERENCE TO RELATED APPLICATIONS

This application claims the benefit of U.S. Provisional Application No. 62/369,158, filed Jul. 31, 2016. The disclosure of the prior application is considered part of and is incorporated by reference in the disclosure of this application.

### BACKGROUND

Presently, laser light is employed in a number of therapeutic applications in mammals. For example, light therapy is commonly used for pain management, to reduce inflammation, and to stimulate photo-biological response to enhance physiological reactions. Typically, appliances and systems used in light therapy applications employ semiconductor Light Emitting Diodes (LEDs) and/or edge-emitting semiconductor lasers to generate optical outputs at wavelengths in the visible and/or near infrared spectral regions.

Generally, light therapy processes require the non-invasive application of light to the skin of the patient proximate to a treatment area at a sufficient energy and wavelength configured to generate the desired therapeutic response. Ideally, the wavelength and power of the light incident on skin of the patient is sufficient to initiate photo-stimulation while not resulting in dermal or sub-dermal ablation or undesirable heating of the tissue. Presently, light therapy systems utilize a large treatment device which is either strapped to the patient or held by a healthcare provider proximate to the area of treatment. Typically, the patient is required to remain stationary during the treatment process, which may range from several minutes to hours.

While presently available light therapy systems have proven somewhat useful in the past, a number of shortcomings have been identified. For example, presently available systems require the patent to remain substantially stationary and immobile during treatment procedures. As such, this inconvenience may result in the patient foregoing needed treatment. Moreover, requiring a human patient to remain stationary during treatment may pose a substantial inconvenience; however, requiring other mammals to remain stationary during treatment may prove difficult if not impossible without sedation or other means. Further, presently available systems tend to be large, expensive systems more adapted for use in professional healthcare facilities.

In light of the foregoing, there is on ongoing need for less expensive light therapy systems that are adapted to be worn by the patient, without requiring the patient to be immobile.

### SUMMARY

Light therapy systems described herein are configured to by worn by or otherwise affixed to the user so that the user is ambulatory while receiving light therapy. In some implementations, one or more physiological sensors are included in the systems such that delivery of the light therapy is effected by physiological parameters of the user detected by the physiological sensors. Moreover, in some implementations the apparatuses described herein also deliver compression therapy in addition to the light therapy.

In one aspect, this disclosure is directed to a wearable apparatus for providing light therapy. The wearable apparatus for providing light therapy includes: (i) a device body wearable by a user such that the user is ambulatory while wearing the device body; (ii) a light source coupled to the device body and configured to output one or more therapeutic optical signals to a bodily treatment area of the user; (iii) a circuit coupled to the device body and comprising a controller and a power source, the controller configured to provide control signals to control the light source; and (iv) a physiological sensor in communication with the controller such that the control signals are effected by physiological parameters of the user detected by the physiological sensor.

Such a wearable apparatus for providing light therapy may optionally include one or more of the following features. The physiological sensor may comprise a temperature sensor configured to measure dermal or sub-dermal tissue temperature of the user. The physiological sensor may comprise an accelerometer configured to measure muscle movements of the user. The physiological sensor may comprise a pulse oximetry sensor configured to measure blood or tissue oxygenation of the user. The power source may be a battery. The battery may be a rechargeable battery. The wearable apparatus may also include a thermoelectric generator configured for converting body heat of the user into electrical current for charging the rechargeable battery. The circuit may also include a wireless interface to facilitate wireless communications between the controller and an external controller. The wearable apparatus may also include the external controller. The external controller may be a smart phone. The device body may be configured in a wearable form selected from a group consisting of: a wrist brace, a knee brace, an ankle brace, a foot brace, a shirt, a pair of shorts or pants, a hat, and a shoe.

In another aspect, this disclosure is directed to a wearable apparatus for providing compression therapy and light therapy to a user. The wearable apparatus includes: (a) a device body wearable by the user, the device body defining one or more chambers that are configured to be pressurized; (b) a light source coupled to the device body and configured to output one or more therapeutic optical signals to a bodily treatment area of the user; and (c) a controller system configured to: (i) supply pressurized fluid to the one or more chambers and (ii) provide control signals to control the light source.

Such a wearable apparatus for providing compression therapy and light therapy to a user may optionally include one or more of the following features. The device body may be configured in a wearable form selected from a group consisting of: a leg wrap and an arm wrap. The light source may be coupled on an inner surface of the device body such that the light source is abutted to a skin surface of the user while the user wears the device body. At least one of the one or more chambers may comprise a visually transparent portion. The light source may be coupled to the device body such that the optical signals from the light source pass through the visually transparent portion and through the pressurized fluid prior to reaching the user. The light source may be repositionable by the user to two or more locations where the light source can be coupled to the device body. The wearable apparatus may also include a physiological sensor in communication with the controller system such that the control signals are effected by physiological parameters of the user detected by the physiological sensor. The device body may define two or more separate chambers that can be pressurized at respective pressures that differ from each other. The controller system may be configured to supply pressurized fluid to each of the two or more separate chambers separately and at differing pressures. The controller system may be configured to control the temperature of the pressurized fluid supplied to the one or more chambers.

In another aspect, this disclosure is directed to a method of treating a subject with compression therapy and light therapy using a wearable apparatus. The method includes: (i) supplying, by a controller system of the wearable apparatus, a pressurized fluid to a chamber defined by a device body of the wearable apparatus while the subject is wearing the device body such that a body portion of the subject is compressed by pressurization of the chamber; and (ii) while the body portion of the subject is compressed, outputting one or more therapeutic optical signals to the body portion of the subject from a light source coupled to the device body, wherein the outputting is controlled by the controller system.

Such a method of treating a subject with compression therapy and light therapy using a wearable apparatus may optionally include one or more of the following features. The pressurized fluid may be water. The pressurized fluid may be air. The pressurized fluid may be temperature controlled by the controller system.

Particular embodiments of the subject matter described in this document can be implemented to realize one or more of the following advantages. In some embodiments, the light therapy system described herein are configured to by worn by or otherwise affixed to the user so that the user is ambulatory while receiving light therapy. In result, the treatments are convenient for the user, and the treatments may be more effective than stationary systems because the treatments can be delivered in real time during activities by the user. In some embodiments, the light therapy systems include one or more physiological sensors that are used as feedback devices such that delivery of the light therapy is effected (e.g., modulated, pulsed, etc.) in response to detected physiological parameters of the user. In some implementations the systems and/or apparatuses described herein also deliver compression therapy in addition to the light therapy. The simultaneous delivery of such a combination of therapies can be especially effective in some cases.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective illustration of an example wearable apparatus for providing light therapy, in accordance with some embodiments.

FIG. 2 is a schematic diagram of an example illumination system that can be used with the wearable apparatuses described herein.

FIG. 3 shows the illumination system of FIG. 2 in a format that is detachably coupleable with a wearable apparatus for providing light therapy in accordance with some embodiments.

FIG. 4 is an illustration of another example wearable apparatus for providing light therapy, in accordance with some embodiments.

FIG. 5 is an illustration of another example wearable apparatus for providing light therapy, in accordance with some embodiments.

FIG. 6 is an illustration of another example wearable apparatus for providing light therapy, in accordance with some embodiments.

FIG. 7 is an illustration of another example wearable apparatus for providing light therapy, in accordance with some embodiments.

FIG. 8 is an illustration of another example wearable apparatus for providing light therapy, in accordance with some embodiments.

FIG. 9 schematically depicts a cross-section of an example wearable apparatus that is providing light therapy to musculature of a user, in accordance with some embodiments.

FIG. 10 is an illustration of an example light therapy system, in accordance with some embodiments.

FIG. 11 is a cross-sectional depiction of the light therapy system of FIG. 10.

FIG. 12 depicts example circuitry of the light therapy system of FIG. 10.

FIG. 13 schematically depicts an example treatment system, in accordance with some embodiments.

FIG. 14 schematically depicts another example treatment system, in accordance with some embodiments.

FIGS. 15 and 16 are illustrations of another example wearable apparatus for providing light therapy, in accordance with some embodiments.

FIG. 17 is an illustration of another example wearable apparatus for providing light therapy, in accordance with some embodiments.

FIGS. 18 and 19 schematically depict a cross-section of an example wearable apparatus that is providing light therapy to a treatment area of a user, in accordance with some embodiments.

FIG. 20 is an illustration of another example wearable apparatus for providing light therapy, in accordance with some embodiments.

FIG. 21 is an illustration of another example wearable apparatus for providing light therapy, in accordance with some embodiments.

FIG. 22 is an illustration of another example wearable apparatus for providing light therapy, in accordance with some embodiments.

FIG. 23 is an illustration of another example light therapy system, in accordance with some embodiments.

FIG. 24 is a side view of a portion of the light therapy system of FIG. 23.

FIG. 25 is an illustration of another example light therapy system, in accordance with some embodiments.

FIG. 26 is a side view of a portion of the light therapy system of FIG. 25.

FIG. 27 schematically depicts a cross-section of another example light therapy system that includes a detachable engaging member, in accordance with some embodiments.

FIG. 28 is an illustration of another example wearable apparatus for providing light therapy and that also includes a fluid pressurization system for providing compression therapy, in accordance with some embodiments.

FIG. 29 is an illustration of another example wearable apparatus for providing light therapy and that also includes a fluid pressurization system for providing compression therapy, in accordance with some embodiments.

Like reference numbers represent corresponding parts throughout.

### DETAILED DESCRIPTION

This document describes various embodiments of light therapy systems configured to be worn by or otherwise affixed to the patient. Moreover, unlike prior art systems, the present light therapy systems disclosed herein may be worn by a moving patient while receiving treatment. In other words, the user can be ambulatory while wearing the light therapy systems.

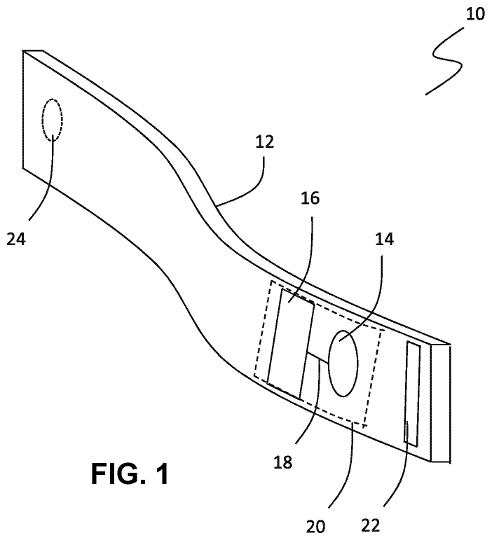

In general, the light therapy system disclosed herein utilizes at least one semiconductor light source configured to deliver at least one therapeutic optical signal to an area of treatment. As shown in FIGS. 1-3, in one embodiment the light therapy system 10 includes at least one device body 12. The device body 12 may be formed in any variety of shapes and sizes. Further, in one embodiment, the device body 12 may be manufactured from polymer material. Exemplary polymer materials include, without limitations, polyimide, neoprene, polyurethane, polyimide, nylon, and the like. Optionally, the device body 12 may be manufactured from a variety of materials, including, without limitations, polymers, natural fibers (e.g. wool, cotton, bamboo, etc.), silicon, elastomers, and the like. Optionally, the device body 12 may include one or more light delivery devices integrated therein or attached thereto. For example, the device body 12 may include one or more fiber optic devices integrated therein. In another embodiment, one or more light guides or similar conduits may be positioned on, coupled to, or otherwise in communication with the device body 12.

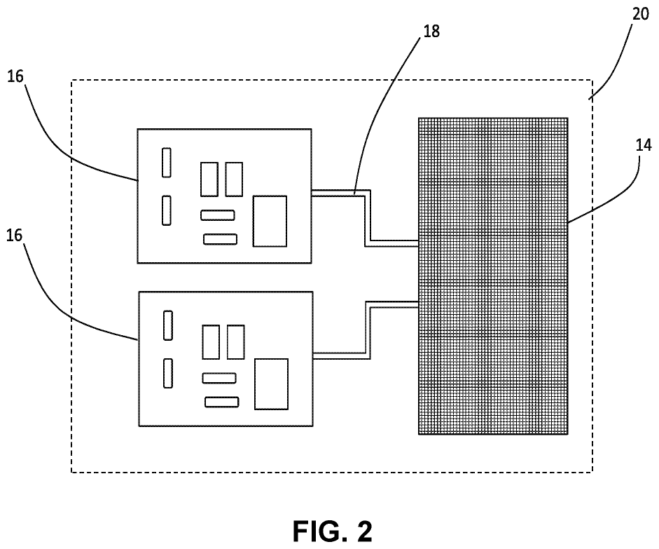

Still referring to FIG. 1-3, at least one semiconductor light source 14 is coupled to device body 12. In one embodiment the light source 14 comprises at least one light emitting diode (hereinafter LED). In an alternate embodiment, the light source 14 comprises at least one laser diode. Exemplary laser diodes configured for use with the present system include, without limitations, edge-emitting laser devices, vertical cavity surface emitting laser devices (hereinafter VCSELs) and the like. Optionally, as shown in FIG. 2, the light source 14 may comprise an array of one or more LEDs or LED die, super-luminescent diode (SLDs), laser diodes or die, or both. Further, in the illustrated embodiment, the light source 14 comprises a LED, a laser diode, or both. For example, LEDs and VCSELs can be fabricated as compact, monolithic arrays of individual emitters to increase the total available power in operation as an ensemble surface-emitting light source. In such cases the individual emitters within an array can be electrically connected to facilitate electrical control of the ensemble as well as integration into flexible/stretchable electronic circuits. Multiple arrays could be similarly connected for ensemble operation and control. Optionally, the light source 14 need not include surface emitting devices. For example, the light source 14 may include one or more fiber optic lasers or fiber optic devices configured to deliver a therapeutic signal to various treatment areas. In another embodiment, one or more light guides or similar conduits may be positioned on, coupled to, or otherwise positioned to be in optical communication with at least one light source 14. For example, one or more light guides may be affixed to at least one of the device body 12 and/or the light source 14 and configured to efficiently deliver light from the light source 14 to an area of interest. Further, the light source 14 may include any variety of light sources.

In some applications, semiconductor light sources are particularly well suited because of a combination of attributes including: high power-to-volume and high power-to-mass; low voltage and low power requirements; efficient conversion of electrical power to light; compatibility with flexible/stretchable electronic circuits and circuit assemblies; ability to operate at wavelengths of interest for light therapy; reliability (e.g., in terms of expected hours of operation, durability); maturity of the technology and associated means of manufacturing; low cost per unit of light power (e.g., dollars per delivered Watt). In addition, semiconductor light sources offer high spatial coherence, facilitating illumination of remote target areas with minimal or no refractive optics. This is especially true of VCSEL versus edge emitting laser. In addition, these sources have high spectral coherence, concentrating light energy at wavelengths of particular interest for specific light therapy applications. Among semiconductor light sources, those based on III-V compounds including both Gallium and Arsenic are the most commonly used for light therapy applications because of their high efficiency (conversion of electrical power to optical power), spectral compatibility with light therapy applications and low cost.

Still referring to FIG. 1-3, in one embodiment the light source 14 is configured to emit at last one therapeutic optical signal having a wavelength in a range from about 400 nm to about 1500 nm. For example, in one embodiment, the light source 14 is configured to output at least one therapeutic optical signal having a wavelength in a range from about 600 nm to about 1100 nm. In another embodiment, the light source 14 is configured to output at least one therapeutic optical signal having a wavelength in a range from about 700 nm to about 1050 nm. In another embodiment, the light source 14 is configured to output at least one therapeutic optical signal having a wavelength in a range from about 780 nm to about 1000 nm. In yet another application, the light source 14 is configured to output at least one therapeutic signal having a wavelength in a range of about 700 nm to about 800 nm. Optionally, a light source 14 may be configured to output multiple optical signals at a single wavelength or a narrow wavelength range. In another embodiment, the light source 14 may be configured to output any number of optical signals at different wavelengths. For example, the light source 14 may be configured to output a first therapeutic optical signal at a first wavelength and at least a second therapeutic optical signal at at least a second wavelength. Further, the light source 14 may be configured to output a continuous wave optical signal, a pulsed optical signal, and/or both. Further, the light source 14 may include one or more optical elements positioned thereon or proximate thereto to condition or otherwise modify the therapeutic light emitted therefrom. For example, the light source 14 may include one or more filters, gratings, lenses and the like positioned thereon or proximate thereto. For example, the light source 14 may include one or more optical metamaterials in optical communication therewith. Exemplary metamaterials include, without limitations, one or more ENZ (epsilon near-zero) metamaterials thereby permitting the output of the light source 14 to be widely tunable over a desired range (e.g. all visible wavelengths).

As shown in FIG. 1-3, the light therapy system 10 includes at least one circuit 16 in electrical communication with the light source 14. In one embodiment, the circuit 16 is configured to provide power to the light source 14. In another embodiment, the circuit 16 is configured to provide data to and receive data from the light source 14. Optionally, the circuit 16 may include one or more semiconductor devices, chips, sensors, controllers, processors, power supplies, batteries, energy sources, voltage regulators, current regulators, user interfaces, display devices, communication devices, wireless communication interfaces, user interfaces, wireless devices, MEMS devices, lab-on-a-chip systems, electricity generators (e.g., thermoelectric generators using the Peltier effect to generate electrical energy to recharge a battery from the user's body heat), and the like. For example, in some embodiments, the circuit 16 includes one or more physiological sensors configured to provide biological information and/or data received from the treatment area of the user. Optionally, the biological information received from the physiological sensors maybe used to vary the treatment parameters, such as the duration of and/or frequency of the treatment, wavelength, pulse length, intensity of the illumination, pulse repetition rate, and the like. In addition, the circuit 16 may include one or more controllers configured to provide information, data, and/or one or more control signals to and receive information, data, and/or one or more control signals from one or more bio-medical sensors (e.g., physiological sensors), controllers, and the like positioned external the body of the user and/or within the body of a user. For example, in some embodiments the circuit 16 may be in communication with at least one external controller (e.g. a smartphone, handheld device, computer, and the like) and at least one sensor or similar device positioned on or within the user. As such, the circuit 16 may act as a conduit configured to provide information to and receive information from the external control device and the sensor wirelessly and/or via a conduit. For example, the circuit 16 may be configured to provide and receive data from at least one of the light source 14, control pumps, drug delivery systems, pacemakers, and the like positioned on or within the body of a patient or user.

In addition, any number of additional sensors may be in communication with or included on the circuit 16. Exemplary additional sensors include, without limitation, flow sensors, oxygenation sensors, pulse oximetry sensors, tissue temperature sensors, accelerometers, force meters, and the like. In one embodiment, the light therapy system 10 includes one light source 14 and one circuit 16. Optionally, the light therapy system 10 may include a single light source 14 in communication with multiple circuits 16. In another embodiment, the light therapy system 10 includes multiple light sources 14 in communication with a single circuit 16. Further, the light therapy system 10 may include multiple light sources 14 in communication with multiple circuits 16. Further, the circuit 16 may include one or more integrated circuit devices, flexible circuits, and/or assemblies of integrated circuits and/or flexible circuits. Optionally, the circuit 16 may include one or more processors with configured to be in communication at least one external controller (not shown). Exemplary external controllers include, for example, computers, handheld devices such as smart phones, tablet computers, and the like. As such, at least one external processor may be configured to provide data to and/or receive data from at least one of the light source 14, circuit 16, and/or both via the circuit 16.

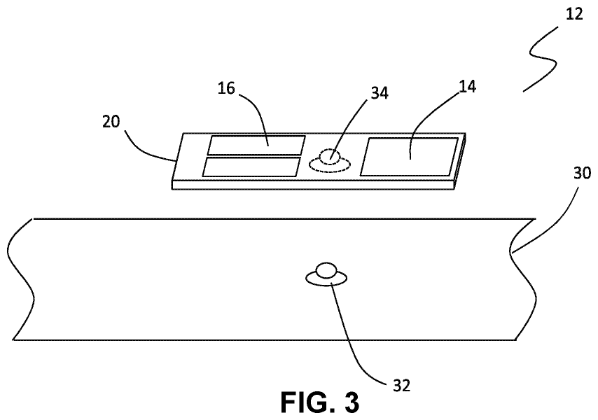

Optionally, as shown in FIGS. 1-3, the light source 14 and the circuit 16 may cooperatively form at least one illumination system body or area 20. In one embodiment, the light source 14 and circuit 16 are integral to the device body 12 of the light therapy system 10. As such, the illumination system body 20 comprises an area containing the light source 14, circuit 16, and the at least one conduit 18 electrically coupling the light source 14 to the circuit 16. In another embodiment, at least one of the light source 14, circuit 16, or both may be detachably coupled to the device body 12. For example, in the embodiment shown in FIG. 3, the illumination system 20 including the light source 14 and circuit 14 are detachably coupled to device body 12. More specifically, the device body 12 may include at least one coupling area 30 formed thereon. In the illustrated embodiment, the coupling area 30 includes at least one coupling feature 32 configured to cooperatively attach to at least one coupling device 34 formed on or otherwise positioned on at least one of the light source 14, circuit 16, and/or illumination system body 20. As such, the illumination system 20 may be removed from the device body 12 in whole or in part, thereby permitting the device body 12 to be washed or otherwise treated (e.g. sterilization, cleaning, and the like) using conventional techniques without damaging the light source 14, circuit 16, conduits 18, and/or illumination system 20. Further, at least one of the light source 14, circuit 16, conduits 18, and/or illumination system 20 may include various housings or other devices to prevent environmental damage to the various components of the light therapy system 10.

Still referring to FIGS. 1-3, in one embodiment the various components of the illumination system 20 may incorporate flex or stretchable electronic circuit technology. More specifically, flexible electronic circuits are by definition compatible with some degree of mechanical deformation. Commonly, flexible circuits are formed by mounting electronic components (e.g. the light source 14 and/or the circuit 16) on flexible substrates, with entire assemblies consisting of one or more (e.g., multi-layer) substrates. As such, at least one of the light source 14, circuit 16, conduit 18, and/or illumination system 20 may be mounted on at least one flexible substrate or may form a flexible electronic circuit. In the present application flexible electronic circuits are particularly useful when intended for deployment within, or as part of, wearable garments and/or accessories (e.g., bracelets). The flexibility of these circuits and the illumination system 20 can be enhanced both by the selection of substrate materials along with the design and selection of embedded components, electrical interconnects and mechanical structures forming the illumination system 20. As such, in one embodiment, the flexible circuits may be integrated into various garments, sleeves, braces, wraps, hats, and the like. Further, the effectively of the light therapy system 10 may also be enhanced by optimizing the design of the light therapy system 10 for use with of one or more garments, accessories, and/or attachment systems or mechanisms (e.g. tape, kinesiology tape, wraps, sleeves, braces, and the like). Optionally, the light therapy system may be configured for use with re-usable garments or disposable garments. For example, in one embodiment the light therapy system 10 is configured for use with compressive garments, thereby providing therapeutic light therapy while simultaneously providing therapeutic compressive support. As such, in addition to providing compressive support, the compressive force of the compressive garment may securely position the light therapy system 10 proximate to a treatment area on a user. In another embodiment, the light therapy system 10 may be configured for use with disposable bandages, wraps, diapers, patches, and the like.

Optionally, one or more portable energy sources may be included within or otherwise coupled to the illumination system 20. For example, in one embodiment at least one power supply system is included within circuit 16 of the illumination system 20. Exemplary power supply systems include, for example, batteries. In one embodiment, the power supply system may be rechargeable. As such, the power supply system may be recharged by conventional means through a wired connection (e.g., utilizing a standardized connector such as a micro USB port) or via some form of wireless inductive charging wherein the receiving antenna and conversion electronics are part of or in communication with the circuit 16. In fact, energy sourced from an external source separate from the light therapy system could be transported wirelessly to directly supply some or all of the devices, components and sub-assemblies of the light therapy system in lieu of batteries. In some embodiments, electricity generators (e.g., thermoelectric generators using the Peltier effect to generate electrical energy to recharge an on-board rechargeable battery from the user's body heat) are included as part of or in communication with the circuit 16.

As shown in FIG. 1, the at least one attachment device 22 is coupled to, positioned on, or otherwise formed in the device body 12 of the light therapy system 10. For example, in one embodiment, the attachment device 22 comprises hook and loop material thereby permitting the user to couple the light therapy system 10 to the body of the user such that the light emitted from the light source 14 will be directed into the body of the user proximate to an area of interest or treatment area. Those skilled in the art will appreciate that any number and variety of attachment devices 22 may be used with the light therapy system 10.

In some embodiments, the light therapy system 10 may further include one or more additional therapeutic systems 24 coupled to the device body 12, light source 14, circuit 16, and/or illumination system 20. Exemplary additional therapeutic systems include, without limitations, muscle stimulations systems, compression systems, chillers/cooling elements, heaters, pumps, drug-delivery systems, pacemakers, diagnostic systems, and the like.

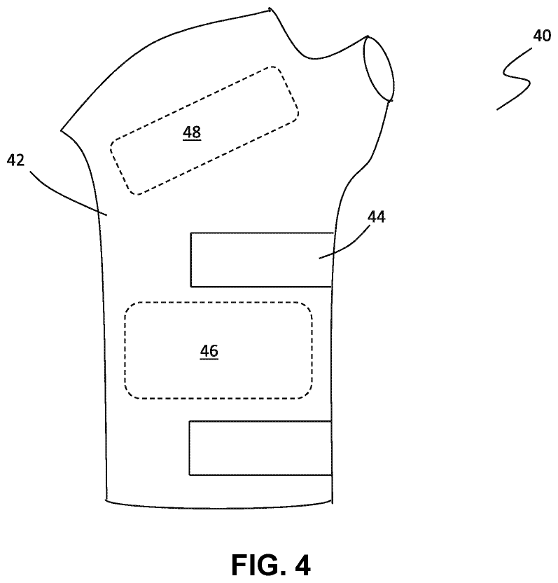

FIGS. 4-10 shows the various embodiments of the light therapy system 10 disclosed herein incorporated into various braces and garments. For example, FIG. 4 shows an embodiment of a skeletal brace 40 configured to be applied to the wrist of a user to deliver therapeutic light to a treatment area. As shown, the brace 40 includes a brace body 42 having at least one attachment device thereon. Further, the brace 40 includes at least one light therapy system coupled thereto or included thereon. In the illustrated embodiment, a first light therapy system 46 and a second light therapy system 48 are positioned on the brace body 44 and configured to direct therapeutic light into the wrist of the user when worn by the user. Unlike prior art systems, the user of the brace 40 shown in FIG. 4, which includes the light therapy system 46, 48, is not required to remain stationary. Rather, the user may preform substantially normal functions required in activities of daily life.

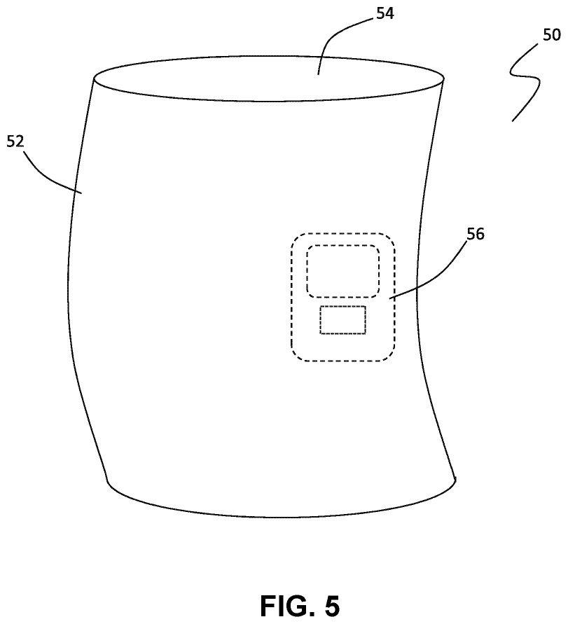

Similarly, FIG. 5 shows an embodiment of brace 50 configured to receive at least one body part therein. For example, the brace 50 shown in FIG. 5 may be configured for use on fingers, wrists, forearms, elbows, biceps, shoulders, triceps, hamstrings, quadriceps, knees, calves, toes, and the like. As shown, the brace 50 includes a brace body 52 defining at least one passage 54. Further, one or more light therapy systems 56 may be coupled to or otherwise positioned on the brace 50 and configured to deliver therapeutic light therapy to an area of interest. In the illustrated embodiment, the brace body 52 may be manufactured from spandex, polyurethane, neoprene, polyimide, or other compressive material and/or material combinations or blends configured to securely position and retain the light therapy system 56 at a desired location.

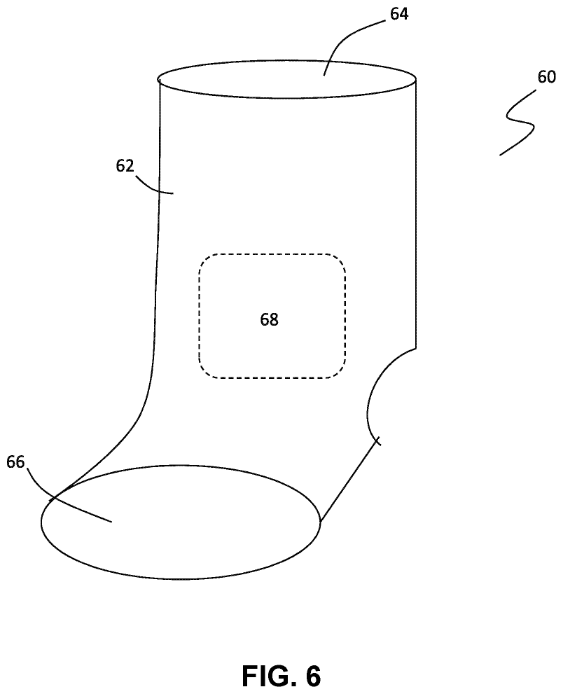

FIG. 6 shows still another embodiment of the light therapy system incorporated into a skeletal brace. As shown, the ankle brace 60 includes brace body 62 defining a first passage 64 sized to receive the low leg of the user and a second passage 66 sized to receive the foot of the user. Further, at least one light therapy system 68 is coupled to or otherwise included on the brace 60 and configured to deliver therapeutic light therapy to an area of interest. Like the previous embodiment, the brace body 62 may be manufactured from spandex, polyurethane, neoprene, polyimide, or other compressive material configured to securely position and retain the light therapy system 68 at a desired location.

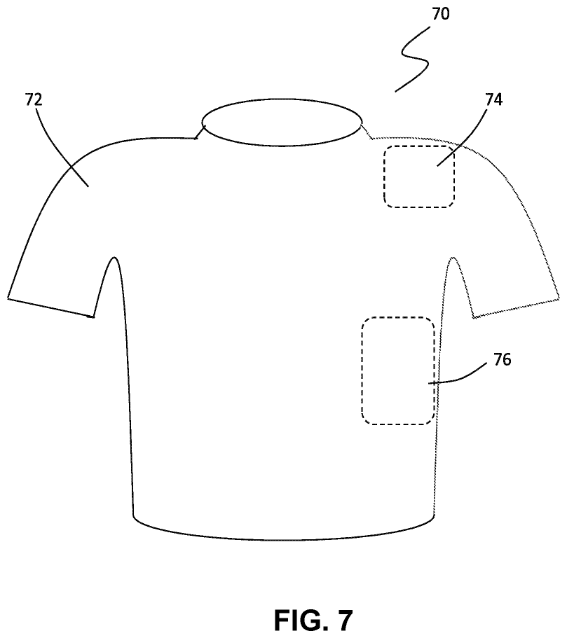

FIG. 7 shows another embodiment of the light therapy system incorporated into a shirt and configured to deliver light therapy to an area of interest located on the upper torso and/or shoulder of the user. As shown, the shirt 70 includes a shirt body 72 having at least one light therapy system coupled thereto or included thereon. In the illustrated embodiment, a first one light therapy system 74 and a second one light therapy system 76 are detachably coupled to the shirt 70. During use, the user would couple the one light therapy systems 74, 76 to the shirt using any variety of attachment devices (See FIG. 1, attachment device 22). Thereafter, the user would initiate the treatment process. For example, in one embodiment, the user would couple the one light therapy system 74, 76 to a user control device (e.g. a handheld device, tablet computer, smartphone, etc.), select the treatment program and parameters from an application, programs or similar control software, and initiate and/or control the treatment process. Thereafter, while the treatment process is occurring, the user may continue his normal activities without being required to remain substantially stationary. In one embodiment, the shirt 70 is manufactured from spandex, polyurethane, neoprene, polyimide, or other compressive material configured to securely position and retain the light therapy system 74, 76 at a desired location.

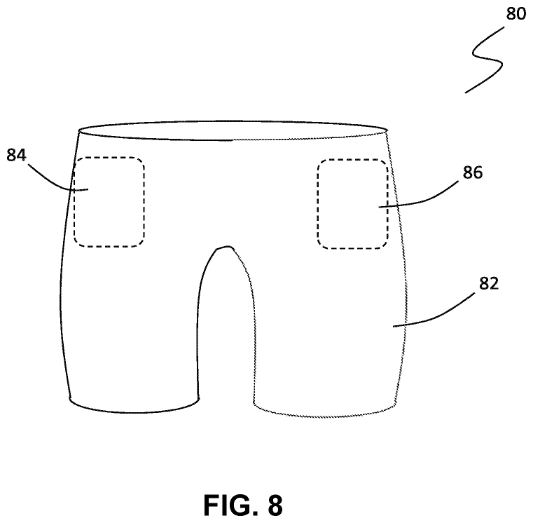

FIG. 8 shows another embodiment of the light therapy system incorporated into a pants and/or shorts and configured to deliver light therapy to an area of interest located on the lower torso of the user. As shown, the shorts 80 include a body 82 having at least one light therapy system coupled thereto or included thereon. In the illustrated embodiment, a first one light therapy system 84 and a second one light therapy system 86 are detachably coupled to the shorts 80. During use, the user would couple the one light therapy systems 84, 86 to the shorts using any variety of attachment devices (e.g., see FIG. 1, attachment device 22). Thereafter, the user would initiate the treatment process. For example, in one embodiment, the user would couple the one light therapy system 84, 86 to a user control device (e.g. a handheld device, tablet computer, smartphone, etc.), select the treatment program and parameters from an application, programs or similar control software, and initiate the treatment process. Like the previous embodiment, while the treatment process is occurring, the user may continue his normal activities without being required to remain substantially stationary. In one embodiment, the shorts are manufactured from spandex, polyurethane, neoprene, polyimide, or other compressive material configured to securely position and retain the light therapy system 84, 86 at a desired location.

As shown in FIGS. 4-8, light therapy system disclosed herein may be attached to or otherwise incorporated into any number of garment, braces, and the like. Exemplary garments include, without limitations, shirts, pants shorts, socks, headbands, hats, caps, gloves, and the like. Similarly, the light therapy system disclosed herein may be include within or coupled to skeletal splints, braces, sleeves, functional orthopedic braces (e.g. CTI-type devices), cervical collars, back braces, and the like. Further, the light therapy system may be included within or coupled to various bandages, wraps, braces and the like used on mammals. As such, the light therapy system disclosed herein may be easily configured to deliver a therapeutic treatment to various limbs, in whole or in part, joints, musculature, and the skeletal structure of a patient.

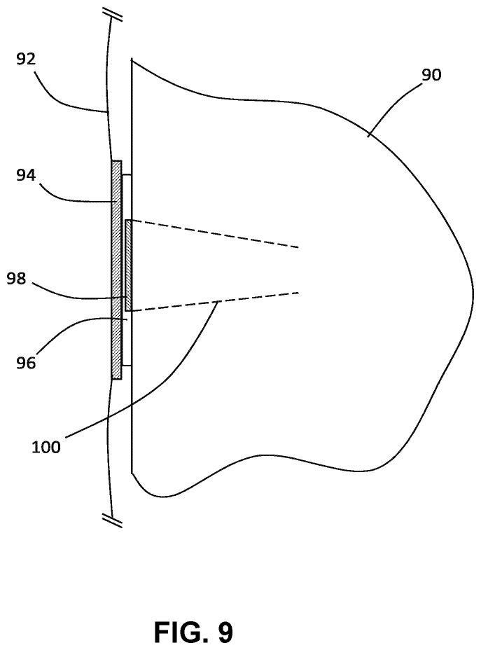

FIG. 9 shows an embodiment of a light therapy system disclosed in the present application during use. As shown, the garment 92 (e.g. shirt) is work by the user. In one embodiment, the garment comprises a compression shirt configured to provide support compressive pressure to the musculature 90 of the user. At least one light therapy system 94 is detachably coupled to the garment 92. As detailed above, the light therapy system 94 includes at least one flexible circuit 96 in communication with at least one light source 98 configured to emit at least one optical signal 100 at a wavelength configured to stimulate a photo-biological response within the musculature 90 and/or other body constituent of the human and/or animal user. As stated above, the compressive force applied by the garment 92 is sufficient to maintain the low-level light therapy system 94 at a desired location during the treatment process.

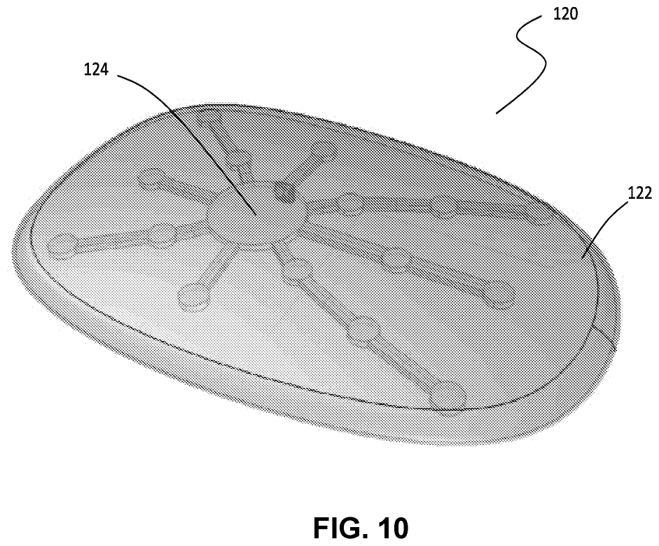

FIGS. 10-12 shows various views of an embodiment of a modular low level light therapy system configured to deliver at least one therapeutic optical signal to an area of treatment. As shown, the modular low level light therapy system 120 includes one or more device bodies or substrates 122 having at least one treatment device circuit 124 positioned thereon or supported the device body 122. In one embodiment, at least one device body 122 used in the modular low level light therapy system 120 comprises a compliant substrate. Optionally, at least one device body 122 used in the modular low level light therapy system 120 may comprise a rigid substrate. Further, in one embodiment, the device body 122 is manufactured from a biologically compatible material, including, without limitations, polyimide, neoprene, polyurethane, polyimide, nylon, and the like. Optionally, the device body 122 may be manufactured from a variety of materials, including, without limitations, polymers, natural fibers (e.g. wool, cotton, bamboo, etc.), silicon, elastomers, and the like. Further, those skilled in the art will appreciate that the device body 122 may be manufactured from any variety of materials permitting washing, sterilization, and the like.

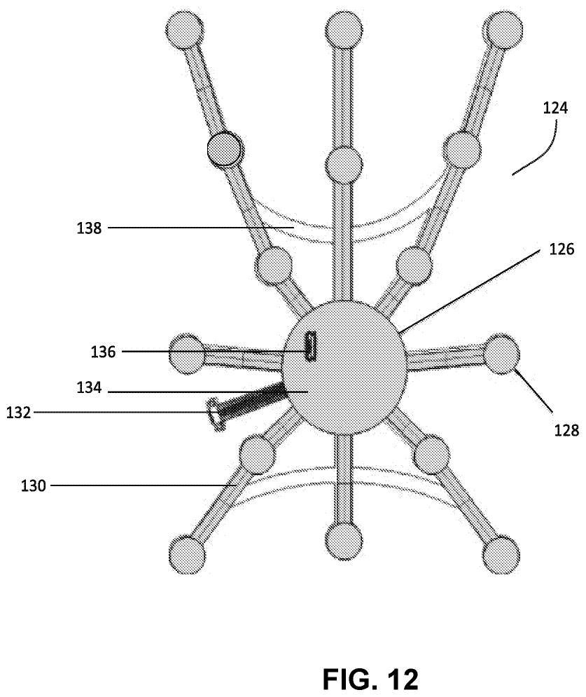

Still referring to FIGS. 10-12, one or more treatment device circuits 124 may be positioned on, integrated within, or otherwise coupled to the device body 122. For example, in one embodiment, at least a portion of the treatment device circuit 124 is positioned within the device body 122. Further, at least a position of the treatment device circuit 124 may traverse through at least a portion of the device body 122. As shown in FIG. 12, in one embodiment, the treatment device circuit 124 includes at least one treatment device controller 126 in communication with at least one low level light source 128. The treatment device controller 126 may be configured to receive data from and transmit data to at least one of controller coupled thereto or in communication therewith. Further, the treatment device controller 126 may be configured to provide energy to the light sources 128 or other device coupled thereto. As such, the treatment device controller 126 may include one or more power supplies, batteries, and the like therein. Further, in one embodiment the light source 128 comprises at least one light emitting diode (hereinafter LED). In an alternate embodiment, the light source 128 comprises at least one laser diode. Exemplary laser diode configured for use with the present system include, without limitations, edge-emitting laser devices, vertical cavity surface emitting laser devices (hereinafter VCSELs) and the like.

Optionally, the light source 128 may comprise an array of one or more LEDs or LED die, super-luminescent diodes (SLDs), laser diodes or die, or both. Further, in the illustrated embodiment, the light source 128 comprises a LED, a laser diode, an edge-emitting laser device, SLDs, or any combination of the aforementioned light sources. For example, LEDs and VCSELs can be fabricated as compact, monolithic arrays of individual emitters to increase the total available power in operation as an ensemble surface-emitting light source. In such cases the individual emitters within an array can be electrically connected to facilitate electrical control of the ensemble as well as integration into flexible/stretchable electronic circuits. Multiple arrays could be similarly connected for ensemble operation and control. Optionally, the light source 128 need not include surface emitting devices. Optionally, the light source 128 may include one or more fiber optic lasers or fiber optic devices configured to deliver a therapeutic signal to various treatment areas. Further, the light source 128 may include any variety of light sources.

As shown in FIG. 12, one or more conduits 130 may couple the treatment device controller 126 to one or more light sources 128. In one embodiment, the conduits 130 comprise flexible devise configure to compliantly couple the various components of the treatment device circuits 124 together electrically. Further, the treatment device circuit 124 may include one or more sensors, user-interface devices, or other component 132 configured to receive or provide information to and from the treatment device controller 126. For example, in one embodiment, the sensor 132 may comprise one or more thermocouples configured to measure dermal or sub-dermal tissue temperature. In another embodiment, the sensor 132 may comprise one or more accelerometers configured to measure tissue movement, muscle extensions/contraction, muscle twitch, muscle movements, and the like. In still another embodiment, the sensor 132 may comprise one or more oxygen sensors configured to measure blood or tissue oxygenation. Optionally, various other sensors may be used in the present system, including, without limitations, heart rate sensors, pulse oximetry sensors, blood pressure sensors, respiration sensors, GPS devices, EKG devices, ECG, devices, and other sensors know in the art. Like the light sources 128, the sensors 132 may be coupled to the treatment device controller 126 via at least one conduit 130.

Still referring to FIG. 12, at least one coupler 136 may be positioned on or proximate to the body 134 of the treatment device controller 126. In one embodiment, the coupled 136 comprises a micro-USB coupler, although those skilled in the art will appreciate that any variety of coupler 136 may be used with the present system. Further, optionally, one or more support members 138 may be formed on or otherwise coupled to at least one of the treatment device controller 126, light sources 128, conduit 130, sensors 132, treatment device controller body 134, and coupler 136. In an alternate embodiment, the treatment device body 124 may be manufactured without support members 138.

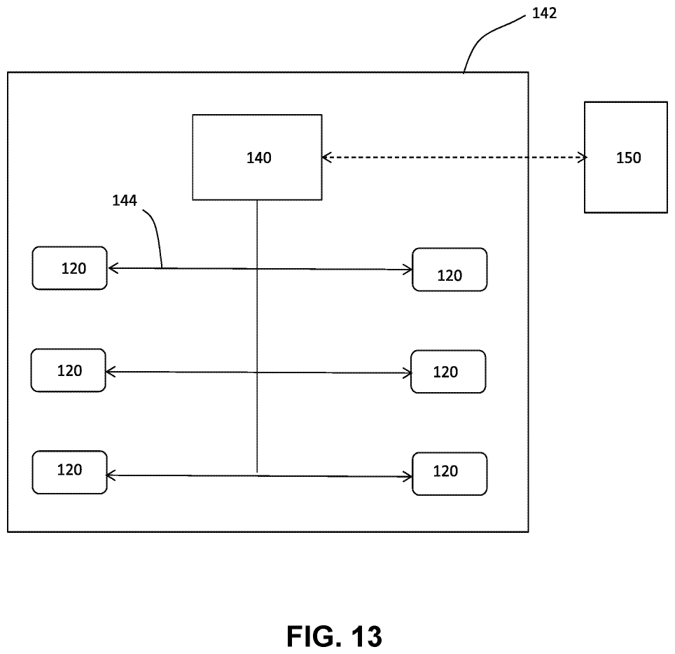

FIG. 13 shows schematically one embodiment of the therapy system which incorporates multiple modular low level light therapy systems 120 to provide therapeutic treatment or to aid in recovery of mammals. As shown in FIG. 13, in one embodiment the treatment system 142 includes one or more modular low level light therapy systems 120 coupled to at least one therapy system controller 140 via at least one conduit 144. In the illustrated embodiment, six (6) modular low level light therapy systems 120 are used in the treatment system 142, although those skilled in the art will appreciate that any number of modular low level light therapy systems 120 may be used.

Further, as shown in FIG. 13, in one embodiment, the treatment system controller 140 detachably coupled to the at least one of the modular low level light therapy system 120, the conduit 144, or both. For example, in one embodiment, the treatment system controller 140 may comprise a detachable dongle thereby permitting the treatment system controller 140 to be selectively attached to and detached from the treatment system 142. Optionally, the treatment system controller 140 need not be detachable from the treatment system 142.

Still referring to FIG. 13, in one embodiment, the treatment system controller 140 is configured to receive data from and provide data to at least one of the external controller 150, modular light therapy systems 120, or both. Further, the treatment system controller 140 may include at least one power supply, battery, or the like therein, the power supply configured to provide power to the modular light therapy systems 120 of the treatment system 142 via the conduit 144. For example, in one embodiment, the treatment system controller 140 includes at least one rechargeable battery therein, thereby permitting the user to selectively detach the treatment system controller 140 from the treatment system and recharge the battery located therein.

Optionally, the treatment system controller 140 may include any number of other devices therein, including, without limitations, various sensors, accelerometers, heart rate monitors, blood pressure monitors, oxygenation monitors, temperature sensors, heating devices, user interface devices, displays, GPS devices, tactile alert devices, audio devices, one or more semiconductor devices, processors, power supplies, voltage regulators, current regulators, communication devices, wireless devices, MEMS devices, lab-on-a-chip systems, and the like. For example, in one embodiment, the treatment system controller 140 may include one or more audio or tactile alert devices configured to alter the user when treatment has initiated or been completed, when power resources are low, and the like. In another embodiment, the treatment system controller 140 may include one or more accelerometers therein, the accelerometers configured to measure muscle twitch and the like.

As shown in FIG. 13, at least one conduit 144 may be used to couple the modular light therapy systems 120 to the treatment system controller 140. In one embodiment, the conduits 144 are configured to have at least one modular light therapy system 120, treatment system controller 140, or both detachably coupled thereto. Further, in one embodiment, the conduits 144 comprise flexible/stretchable conduits configured to be integral to at least one fabric or garment. Exemplary garments include, without limitations, shirts, pants shorts, socks, headbands, hats, caps, gloves, and the like. For example, the conduit 144 may be woven into, or otherwise not detachably coupled to the garment. In another embodiment, at least one of the treatment system 142, treatment system controller 140, conduit 144, and/or modular light therapy system 120 may be positioned proximate to an area of treatment by at least one of a compressive wrap, tape, or strap, compressive garment, compressive sleeve, or biologically-compatible adhesive. In another embodiment, the flexible conduit 144 may be non-detachably coupled to garment.

Similarly, the conduit 144 may be integral to or coupled to skeletal splints, braces, sleeves, functional orthopedic braces (e.g. CTI-type devices), cervical collars, back braces, and the like. Further, the conduit 144 may be included within or coupled to various bandages, wraps, blankets and the like used on mammals. Optionally, the conduit 144 may be detachable coupled to various skeletal splints, braces, sleeves, cervical collars, back braces, bandages, wraps, blankets and the like.

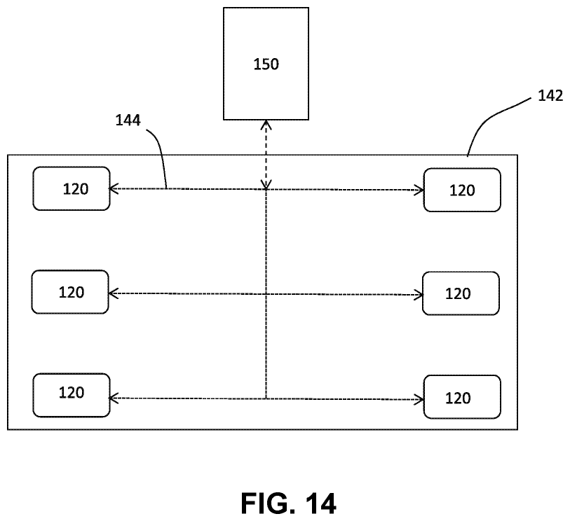

FIG. 14 shows another embodiment of a therapy system which incorporates multiple modular light therapy systems 120 to provide therapeutic treatment or to aid in recovery of mammals. Like the previous embodiment, the therapy system shown in FIG. 14 includes multiple modular light therapy systems 120, although, like the previous embodiment, those skilled in the art will appreciate that the therapy system may be configured to include a single modular light therapy system 120. However, in the present embodiment, the treatment system 142 utilizes a distributed control system, wherein the individual modular light therapy systems 120 cooperatively form a treatment system control architecture thereby forgoing the need for the treatment system controller 140 shown in FIG. 13. As such, the individual modular light therapy systems 120 may be configured to provide data to and receive data from neighboring modular light therapy systems 120, the external controller 150, or both. In one embodiment, the modular light therapy systems 120 may communicate with associated modular light therapy systems 120, the external controller 150, or both wirelessly. In the alternative, the modular light therapy systems 120 may communicate with associated modular light therapy systems 120, the external controller 150, or both via at least one conduit. In an alternate embodiment, the compressive sleeve 120 may be manufacture without a device receiver 162 and device pocket 164. Rather, the compressive force applied by the compressive sleeve 160 may be sufficient to position and retain the modular light therapy system 120 proximate to an area of treatment.

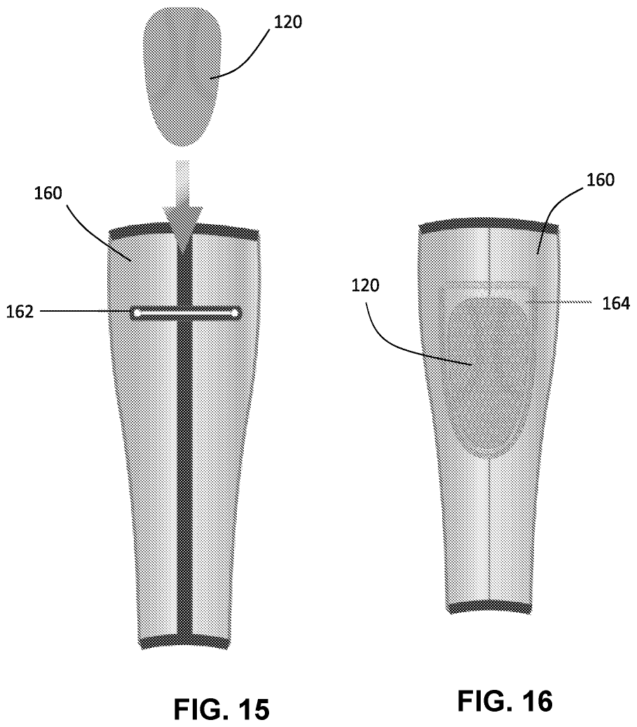

FIGS. 15-22 show various embodiments of the modular light therapy system 120 in use. For example, as shown in FIGS. 15 and 16, show a compressive sleeve 160 configured to receive at least one therein. FIG. 15 shows a perspective view of the sleeve 1609 which includes at least one device receiver 162 sized to receive at least one modular light therapy system 120 therein formed thereon. FIG. 16 shows a cross-sectional view of the sleeve 160 which includes at least one device pocket 164 formed or otherwise attached thereto. In one embodiment, the device pocket 164 is manufactured from a mesh material. In another embodiment, the device pocket 164 is manufactured from any material substantially transparent to an optical signal having a wavelength from about 200 nm to about 2000 nm. The device pocket 164 is in communication with the device receiver 162. During use, the user inserts the modular low level light therapy system 120 into device pocket 164 via the device receiver 162 and dons the sleeve. Thereafter, the positions the sleeve such that the modular light therapy system 120 is positioned proximate to an area of treatment. Finally, the initiate treatment via at least one of the treatment system controller 140, if present, the external controller 150, if present, or both (e.g., see FIGS. 13 and 14).

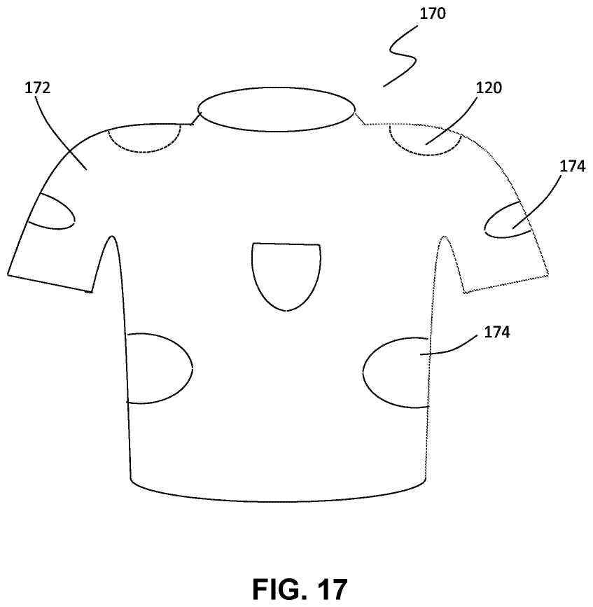

FIG. 17 shows an alternate application of the modular light therapy system 120. As shown in FIG. 17, a shirt 170 having garment body 172 may be configured to receive multiple modular low level light therapy systems 120 therein. As shown, the garment body 172 may include one or more device pockets 174 sized to receive at least one modular low level light therapy system 120 therein. In the alternative, like the previous embodiment, the shirt 170 may be configured to provide sufficient compressive or positioning force to the modular light therapy system 120 to negate the need for device pockets 174. FIG. 17 shows at least one modular light therapy system 120 compressively positioned proximate to an area of treatment, the compressive force being applied to the modular low level light therapy system 120 by the garment body 172.

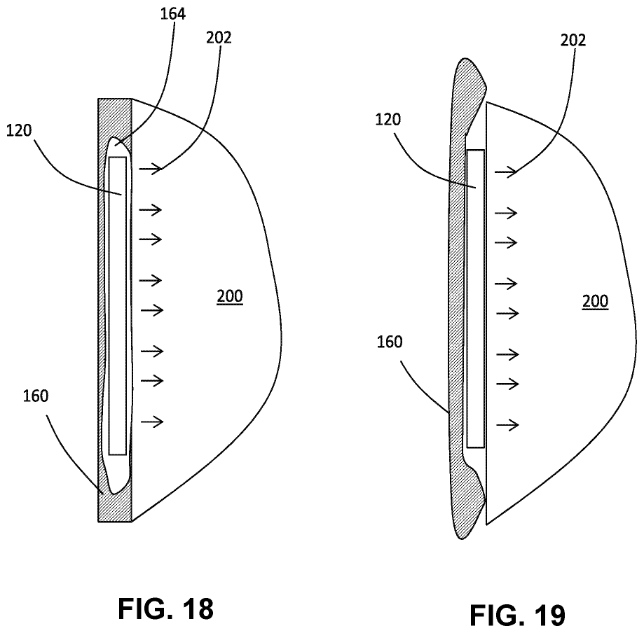

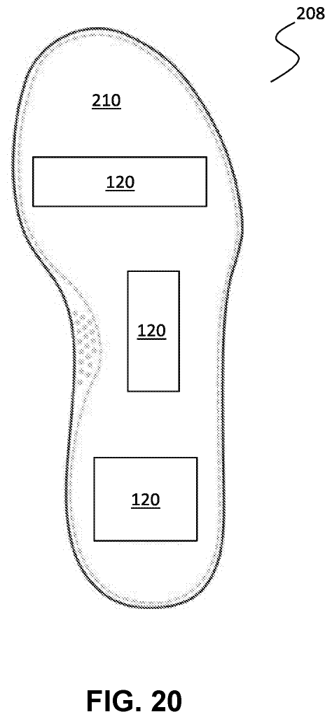

FIGS. 18 and 19 show cross-sectional views of various embodiments of the modular low level light therapy system 120 in use. As shown in FIG. 18, the modular low level light therapy system 120 may be positioned within the device pocket 164 formed within the sleeve 160. See FIGS. 15 and 16. Thereafter, the sleeve 160 may be positioned on the body of the user or patient proximate to an area treatment 200. During use, the therapeutic treatment 202 is applied through the material forming the device pocket 164. In the alternative, FIG. 19 shows an embodiment of modular low level light therapy system 120 positioned on the body of the user or patient proximate to an area treatment 200 and retained in the desired location by the compressive force applied by the sleeve 160. As such, the therapeutic treatment 202 is applied directly to the tissue surface. Those skilled in the art will appreciate that while FIGS. 18 and 19 illustrate the use of the modular low level light therapy system 120 with a sleeve, the modular low level light therapy system 120 may be effectively used with any variety of shirts, pants shorts, socks, headbands, hats, caps, gloves, compressive wraps, tapes, straps, compressive garments, compressive sleeves, skeletal splints, braces, sleeves, functional orthopedic braces (e.g. CTI-type devices), cervical collars, back braces, bandages, blankets and the like used on mammals. For example, FIG. 20 shows an insole 208 having an insole body 210 having one or more modular low level light therapy system 120 positioned therein or coupled thereto. Similarly,

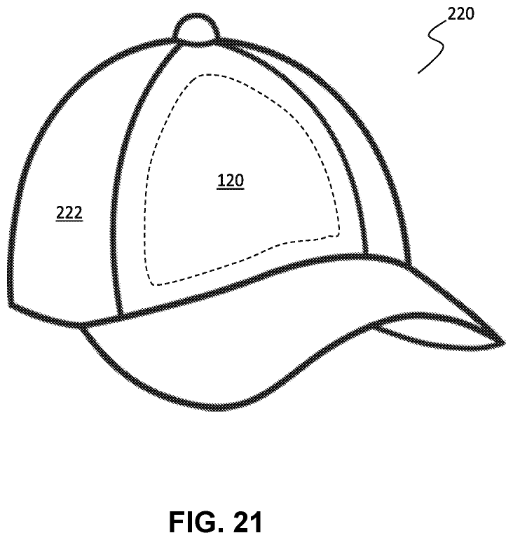

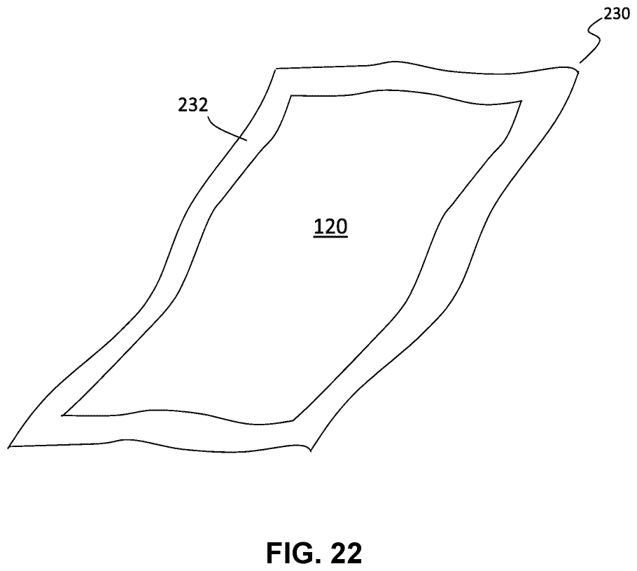

FIG. 21 shows an embodiment of a cap 220 having a cap body 222 having one or more modular low level light therapy system 120 positioned therein or coupled thereto. Further, FIG. 22 shows an embodiment of a blanket 230 having a blanket body 232 having one or more modular low level light therapy system 120 positioned therein or coupled thereto. While some garments described herein are particularly well suited for use on humans, other applications, such as braces, wraps, compressive sleeves, blankets, and the like are applicable for human use as well as many varieties of mammals, including, without limitations, horses, dogs, cats, cows, birds, reptiles, and the like.

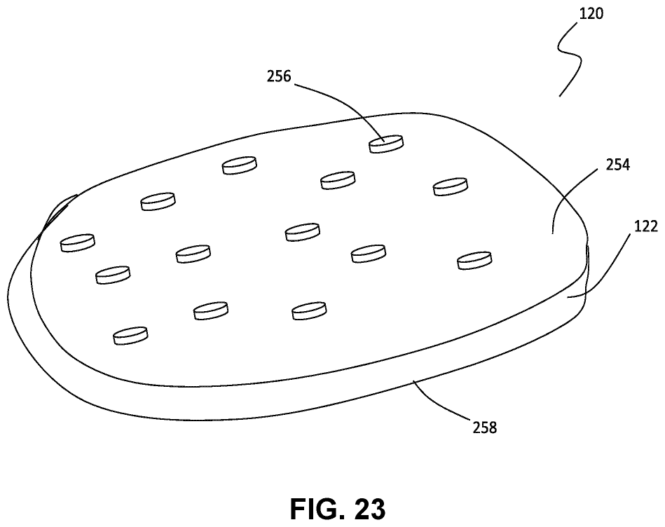

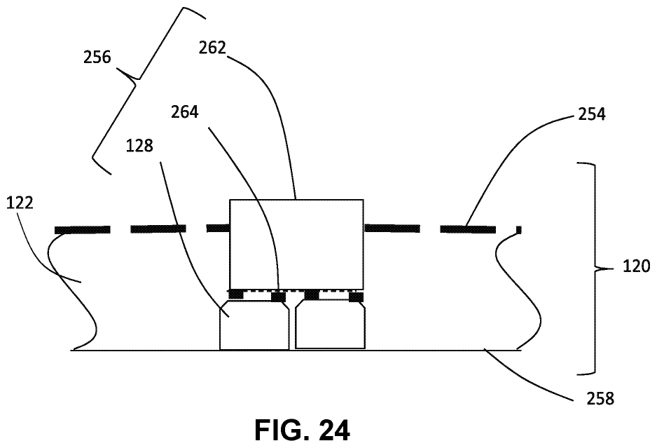

FIGS. 23-27 show various embodiments of a low level light therapy system which include one or more light guides or similar conduits devices. The low level light therapy systems shown in FIGS. 23-27 are analogous to the low level light therapy system 120 shown in FIGS. 10-14, and incorporates the features thereof. As such, common elements and features shown in FIGS. 10-14 and 23-27 share common references numbers. FIGS. 23 and 24 show an embodiment of a low level light therapy system 250. As shown, the low level light therapy system 120 shown in FIGS. 23 and 24 includes at least one device body 122 having one or more treatment circuits 124, light sources 128, and similar components (See FIGS. 10-14) as described above.

Referring again to FIGS. 23 and 24, the low level light therapy system 120 includes at least one treatment surface 254 having one or more light delivery systems, conduits, and/or devices 256 positioned therein or extending therefrom. In the illustrated embodiment, the light delivery system 256 comprises at least one light guide 262 coupled to or positioned proximate to at least one light source 128 such that the light guide 262 acts as a conduit directing at least a portion of the optical signal generated by the light source 128 to an area of interest. In the illustrated embodiment, the light guide 262 is adhesively coupled to the light source 128 using at least one adhesive bond 264. Optionally, the light guide 262 may be coupled to the light source 128 using any variety of mechanisms. For example, the light guide 262 may be coupled to the light source 128 using one or more mechanical couplers or fixtures. Optionally, the light guide need not be coupled to the light source 128. Rather, the light guide 262 may be positioned in close proximity to the light source 128 such that the light guide 262 receives light from the light source 128 and delivers at least one therapeutic optical signal to an area of interest on a user or mammal receiving treatment. In one embodiment, the light guide 262 may be configured to control the beam dimensions and divergence of the light signal emitted by the light source 128 and divergence thereby controlling the distribution of the light signal at the skin. As such, the light guide 262 may be configured to enhance the therapeutic effects of the low level light therapy system. Optionally, the light guide 262 may be configured to reflect and/or scatter light travelling through the light guide 262 off the perimeter surfaces of the light guide 262 so as to enhance the conveyance of light signal there through.

Still referring to FIGS. 23 and 24, in one embodiment, the light guide 262 comprises a cylindrical body having a length from about 1 mm to about 20 mm. In another embodiment, the light guide 262 has a length from about 3 mm to about 10 mm. These include conveying the light in ways to control the beam dimensions and divergence at the skin (or, basically, to intentionally change both to enhance therapeutic effect). The second are the how's. The conduit can work by reflecting and/or scattering light off of the perimeter surfaces of the light guide so as to enhance the conveyance of light through the light guide. Optionally, the light guide 262 may have a length of about 8 mm. Further, the light guide 262 may have a transverse dimension in a range from about 0.1 mm to about 20 mm. For example, in a more specific embodiment, the light guide 262 has a transverse dimension in a range from about 5 mm to about 12 mm. In another embodiment, the light guide 262 has a transverse dimension of about 5 mm. In the illustrated embodiment, a single light guide 262 is coupled to or positioned proximate to a single light source 128.

Optionally, multiple light guides 262 may be coupled to a single light source 128. In the alternative, a single light guide 262 may be coupled to multiple light sources 128. As such, the light guide 262 may be manufactured in any variety of shapes, configurations, lengths, transverse dimensions, and the like. Further, the light guide 262 may be manufactured from any variety of materials. In one embodiment, at least one light guide 262 is manufactured from a polymer material configured to transmit light at a desired wavelength range (e.g., about 400 nm to about 1500 nm) therethrough. In another embodiment, the light guide 262 may be configured to transmit light having a wavelength in a range from about 500 nm to about 900 nm therethrough. Optionally, the light guide 262 may be configured to transmit light having a wavelength in a range from about 700 nm to about 800 nm therethrough. In some embodiments, the light guide 262 may be manufactured from any variety of materials, including, without limitations, polymers, optical crystals, silica-based materials, fiber optic devices, waveguides, and the like. In addition, the light guide 262 may include one or more filters, attenuators, lenses, sensors, thermocouples, and the like incorporated therein or attached thereto. Optionally, the light guides 262 may form rigid bodies, semi-rigid bodies, and/or compliant bodies configured to enhance the delivery of light to an area of interest while simultaneously providing tactile feedback or pressure release therapy to the user.

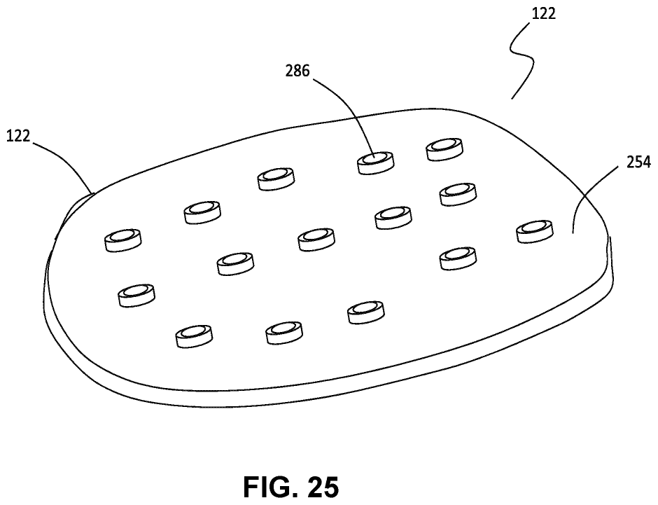

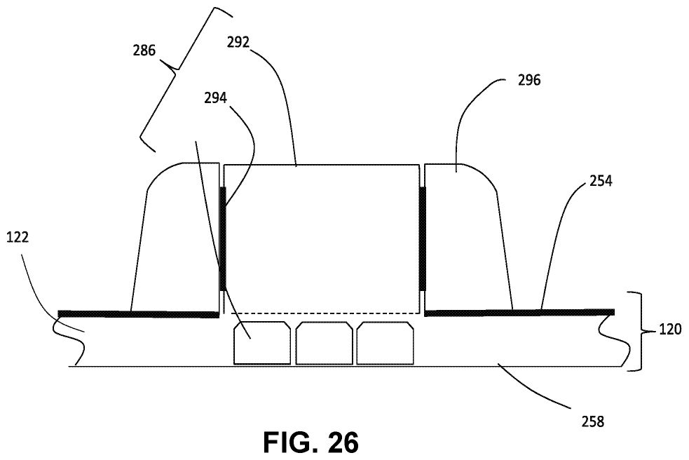

FIGS. 25 and 26 show an alternate embodiment of the low level light therapy system employing light guides as shown in FIGS. 23 and 24. As shown in FIGS. 25 and 26, the light therapy system 120 includes a device body 122 having one or more treatment circuits 124, light sources 128, and similar components (e.g., see FIGS. 10-14) as described above. In addition, the light therapy system 120 includes at least one treatment surface 254 having one or more light delivery systems, conduits, and/or devices 286 positioned therein or extending therefrom. In the illustrated embodiment, the light delivery system 286 comprises at least one light guide 292 coupled to or positioned proximate to at least one light source 128 such that the light guide 292 acts as a conduit directing at least a portion of the optical signal generated by the light source 128 to an area of interest. In the illustrated embodiment, the light guide 292 is positioned within at least one support body 296 coupled to at least one of the treatment surface 254 and/or the light source 128. In the illustrated embodiment, the light guide 292 is adhesively coupled to support body using at least one adhesive bond 294. Optionally, the light guide 262 may be coupled to the support body 296 and/or the light source 128 using any variety of mechanisms. The support body 296 may be configured to support and protect at least one of the light guide 292 and/or the light source 128. In addition, the support body 296 may be configured to support additional components, including, for example, sensors, lenses, filters, massage devices and features, and the like. As such, the support body 296 may be manufactured in any variety of sizes, shapes, transverse dimensions, and the like. Further, the support bodies 296 may comprise rigid bodies, semi-rigid bodies, and/or compliant bodies or features.

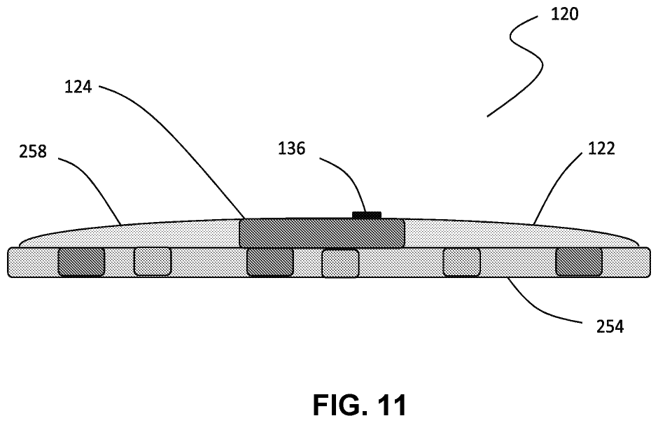

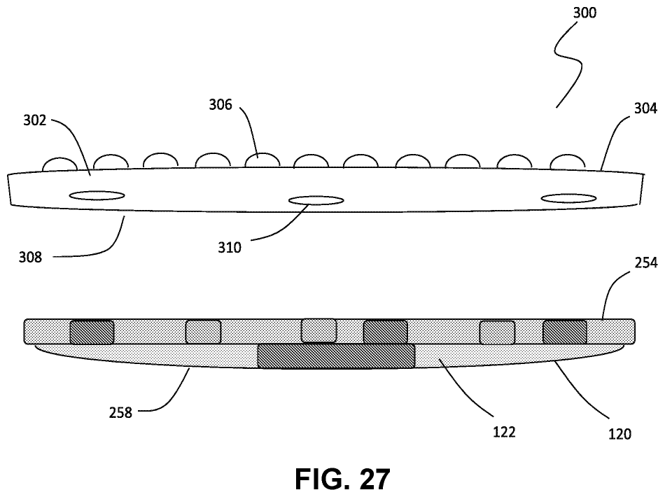

FIG. 27 shows an alternate embodiment of a low level light therapy system having a detachable engaging member. Optionally, the low level light therapy system may be operated without a detachable engaging member. The detachable engaging member 300 is configured to be selectively coupled to and be removed from the device body 122 of the light therapy system shown in FIGS. 10-14. As such, the engaging member 300 includes at least one device body 302 having at least one treatment surface 304 formed thereon. At least one receiving aperture 308 is formed on the device body 302, the receiving aperture 308 configured to receive at retain at least a portion of the device body 122 of the light therapy system 120 therein (see FIGS. 10 and 11). In one embodiment, the receiving aperture 308 is configured to selectively couple the engaging member 300 to the light therapy system 120. As such, the device body 302 may include one or more coupling features 310 formed thereon. Exemplary coupling features include, for example, slip fit members, friction features, tabs, locks, and the like.

Still referring to FIG. 27, at least one light guide system 306 similar to the light guide systems described in FIGS. 23-26 may be positioned on the device body 302. For example, in one embodiment, the light guide system 306 is configured be positioned proximate to the light sources 128 formed on the device body 122 of the light therapy system 120 (See FIGS. 10 and 11). As such, the light guide systems 306 coupled to or otherwise positioned on the treatment surface 304 of the device body 302 may be configured to act as a conduit to transport light emitted from the light therapy system 120 to an area of interest. The detachable engaging member 300 may be configured to be easily removed from the light therapy system 120 to enable washing, cleaning, and/or sterilization of at least one of the detachable engaging member 300 and the light therapy system 120. Further, the easily removable detachable engaging member 300 enables a user to easily tailor the configuration of the light therapy system 120 for a particular use. For example, a first detachable engaging member 300 having a first configuration of light guide systems may be affixed to the light therapy system 120 for a first user. Thereafter, the first detachable engaging member 300 having the first configuration of light guide systems may be removed from to the light therapy system 120 and replaced with a second detachable engaging member 300 having a second light guide system configuration for a second user. The light guide systems may be configured to have any number, length, transverse dimension, and shape light guide systems of components thereof. Further, the detachable engaging member 300 may include any variety of sensors of similar components thereon.

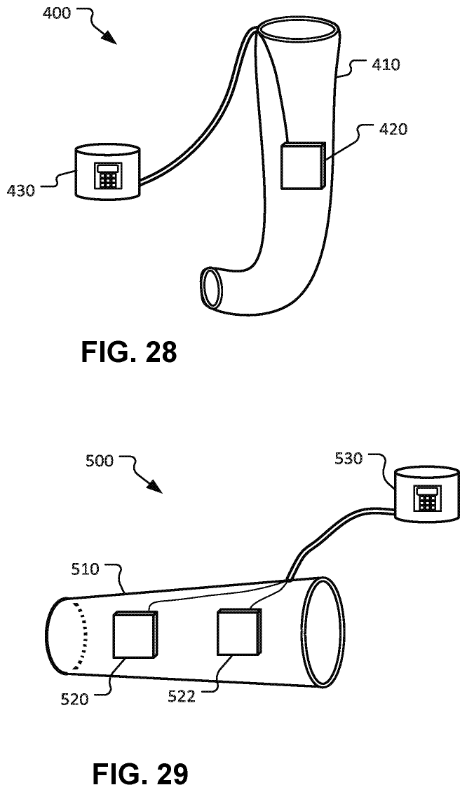

Referring to FIGS. 28 and 29, light therapy (e.g., as described above) can be combined with compression therapy using the example systems 400 and 500. System 400 includes a device body 410 that is configured to receive a subject's leg, or a portion thereof System 500 includes a device body 510 that is configured to receive a subject's arm, or a portion thereof. Other device body configurations are also envisioned, such as, but not limited to, braces (e.g., knee, wrist, ankle, elbow, etc.), wraps, shirts, pants/shorts, blankets, and the like. The device bodies 410 and/or 510 can include one or more zippers, closures, straps, and the like to make donning the device bodies 410 and/or 510 more convenient for the user.

The device bodies 410 and 510 define one or more chambers configured to be pressurized by a fluid such as air or water. When the one or more chambers are pressurized by a fluid, compression therapy is delivered to a subject who is wearing the device body 410 or 510. When the device body 410 and/or 510 includes multiple pressurize-able chambers, the chambers can be pressurized at differing pressures and/or temperatures, or at the same pressure and/or temperature. Accordingly, a virtually infinite number of treatment regimens are possible using the systems 400 or 500.

In some embodiments, the device bodies 410 and 510 include one or more transparent portions that transmit light generated from light sources coupled to the device bodies 410 and 510. Accordingly, such light can pass through one or more walls of device body 410 and/or 510, and through the fluid that pressurizes the chambers of the device bodies 410 and/or 510, to reach the body part of the user. In some embodiments, a substantial entirety of the device bodies 410 and/or 510 are transparent.

The system 400 also includes a light therapy system 420. Similarly, the system 500 includes a first light therapy system 520 and a second light therapy system 522. Accordingly, light therapy (as described above) can be delivered to a subject simultaneously with compression therapy.

In the depicted embodiments, the light therapy systems 420, 520, and 522 are coupled on an outer wall surface of the device bodies 410 and 510 where there are transparent areas of the device bodies 410 and 510. In some cases, one or more of the light therapy systems 420, 520, or 522 are coupled on an inner wall surface of the device bodies 410 and 510 facing toward and adjacent to (e.g., abutting) the user's body part. In some embodiments, the light therapy systems 420, 520, and/or 522 are repositionable to different regions of the device bodies 410 and/or 510 as desired by the user.

One or more light therapy systems can be attached to a device body 410 and/or 510. For example, one, two, three, four, five, six, seven, eight, nine, ten, or more than ten light therapy systems can be included on a single device body 410 and/or 510. In some cases, the user can customize the number of, and/or positioning of, the light therapy systems used in conjunction with a single device body 410 and/or 510.

The systems 400 and 500 also include controller systems 430 and 530 respectively. The controller systems 430 and 530 can provide pressurized fluid (e.g., water, air) to the chambers of the device bodies 410 and 510, and can provide control signals to the light therapy systems 420, 520, and 522. In some embodiments, the controller systems 430 and 530 also can cool and/or heat the pressurized fluid supplied to the device bodies 410 and/or 510.

The controller systems 430 and 530 can be programmable and/or user-adjustable. For example, when a device body 410 and/or 510 has multiple pressurize-able chambers, the controller systems 430 and/or 530 can vary the relative pressures according to a pattern that provides a desired therapeutic effect. In another example, the delivery of light therapy can be coordinated with the delivery of compression therapy in a desired manner or pattern.

In some embodiments, the systems 400 and 500 also include one or more physiological sensors (not shown) as described above. Such sensors can be in communication with the controller systems 430 and/or 530, and the controller systems 430 and/or 530 can modulate the light therapy and/or compression therapy based on signals from the physiological sensors.

The embodiments disclosed herein are illustrative of the principles of the invention. Other modifications may be employed which are within the scope of the invention. Accordingly, the devices disclosed in the present application are not limited to that precisely as shown and described herein.

What is claimed is:

1. A wearable apparatus for providing light therapy, the wearable apparatus comprising:
a device body wearable by a user such that the user is ambulatory while wearing the device body;
a light source coupled to the device body and configured to output one or more therapeutic optical signals to a bodily treatment area of the user;
a circuit coupled to the device body and comprising a controller and a power source, the controller configured to provide control signals to control the light source; and
a physiological sensor in communication with the controller such that the control signals are effected by physiological parameters of the user detected by the physiological sensor.

2. The wearable apparatus of claim 1, wherein the physiological sensor comprises a temperature sensor configured to measure dermal or sub-dermal tissue temperature of the user.

3. The wearable apparatus of claim 1, wherein the physiological sensor comprises an accelerometer configured to measure muscle movements of the user.

4. The wearable apparatus of claim 1, wherein the physiological sensor comprises a pulse oximetry sensor configured to measure blood or tissue oxygenation of the user.

5. The wearable apparatus of claim 1, wherein the power source is a battery.

6. The wearable apparatus of claim 5, wherein the battery is a rechargeable battery and further comprising a thermoelectric generator configured for converting body heat of the user into electrical current for charging the rechargeable battery.

7. The wearable apparatus of claim 1, wherein the circuit further comprises a wireless interface to facilitate wireless communications between the controller and an external controller.

8. The wearable apparatus of claim 7, further comprising the external controller, and wherein the external controller is a smart phone.

9. The wearable apparatus of claim 1, wherein the device body is configured in a wearable form selected from a group consisting of: a wrist brace, a knee brace, an ankle brace, a foot brace, a shirt, a pair of shorts or pants, a hat, and a shoe.

10. A wearable apparatus for providing compression therapy and light therapy to a user, the wearable apparatus comprising:
a device body wearable by the user, the device body defining one or more chambers that are configured to be pressurized;
a light source coupled to the device body and configured to output one or more therapeutic optical signals to a bodily treatment area of the user; and
a controller system configured to: (i) supply pressurized fluid to the one or more chambers and (ii) provide control signals to control the light source.

11. The wearable apparatus of claim 10, wherein the device body is configured in a wearable form selected from a group consisting of: a leg wrap and an arm wrap.

12. The wearable apparatus of claim 10, wherein the light source is coupled on an inner surface of the device body such that the light source is abutted to a skin surface of the user while the user wears the device body.

13. The wearable apparatus of claim 10, wherein at least one of the one or more chambers comprises a visually transparent portion, and wherein the light source is coupled to the device body such that the optical signals from the light source pass through the visually transparent portion and through the pressurized fluid prior to reaching the user.

14. The wearable apparatus of claim 10, wherein the light source is repositionable by the user to two or more locations where the light source can be coupled to the device body.

15. The wearable apparatus of claim 10, further comprising a physiological sensor in communication with the controller system such that the control signals are effected by physiological parameters of the user detected by the physiological sensor.

16. The wearable apparatus of claim 10, wherein the device body defines two or more separate chambers that can be pressurized at respective pressures that differ from each other.

17. The wearable apparatus of claim 16, wherein the controller system is configured to supply pressurized fluid to each of the two or more separate chambers separately and at differing pressures.

18. The wearable apparatus of claim 16, wherein the controller system is configured to control the temperature of the pressurized fluid supplied to the one or more chambers.

19. A method of treating a subject with compression therapy and light therapy using a wearable apparatus, the method comprising:
supplying, by a controller system of the wearable apparatus, a pressurized fluid to a chamber defined by a device body of the wearable apparatus while the subject is wearing the device body such that a body portion of the subject is compressed by pressurization of the chamber; and
while the body portion of the subject is compressed, outputting one or more therapeutic optical signals to the body portion of the subject from a light source coupled to the device body, wherein the outputting is controlled by the controller system.

20. The method of claim 19, wherein the pressurized fluid is water or air.

21. The method of claim 19, wherein the pressurized fluid is temperature controlled by the controller system.
